(19) United States
(12) Patent Application Publication
(10) Pub. No.: US 2005/0090708 A1
(43) Pub. Date: Apr. 28, 2005

(54) Tapeless device for retaining the foreskin of a circumcised penis over the glans

(76) Inventor: Ron J. Low, Northbrook, IL (US)

Correspondence Address: Ron J. Low, 2020 Landwehr, Northbrook, IL, 60062, US

(21) Appl. No.: 10/946,862

(22) Filed: Sep. 22, 2004

Related U.S. Application Data
(60) Provisional application No. 60/503,815, filed on Sep. 22, 2003.

(51) Int. Cl. A61F 5/00

(52) U.S. Cl. 600/38

(57) ABSTRACT

This device gently holds the foreskin of a circumcised penis in a fully extended state, so that the glans may be covered as much as possible with the user's natural skin. The device consists of a shell of silicone rubber in the shape of a truncated cone. Through novel geometry and construction, the device is able to hang on to the user's penis with no adhesive, allowing for convenient and discrete coverage of the glans without the clumsy, bulky, and messy fixtures and techniques associated with other foreskin restoration methods. This covering of skin keeps the glans and inner skin healthy, supple, and sensitive. Also claimed are complementary spacer devices for adding tension to the skin without externally applied force, as well as fixtures for manufacture of the devices.

### CROSS-REFERENCE TO RELATED APPLICATIONS

This application should benefit from an earlier Provisional Application: Application No. 60/503,815 Filing Date: Sep. 22, 2003

### STATEMENT REGARDING FEDERALLY SPONSORED RESEARCH OR DEVELOPMENT

Not applicable

### BACKGROUND OF THE INVENTION

This invention is a skin stretching device intended to improve the vitality of the human penis.

In America and other places, the foreskin of the penis is often removed through a procedure called circumcision. This is usually carried out on an infant unable to consent to the amputation of part of his penis.

In recent years, it has become more and more clear that circumcision removes valuable tissue while providing few, if any, benefits to the victim. Statements by national medical organizations the world over make it clear there is no medical justification for routine circumcision. Aside from the risks of a surgical procedure with no benefits, common drawbacks of circumcision include the desensitizing of the glans (which is protected by the foreskin of a normal penis), loss of numerous nerve endings and unique pleasure-receptor tissues, and permanent malformation and malfunction of the penis due to excess skin removal, irregular scar tissue, or unintended injury to parts of the penis.

The glans and “inner” skin (the area on the inner side of the foreskin in a normal penis) are normally covered by the foreskin, which protects these parts from drying and abrasion. Once the foreskin is removed, these parts loose their suppleness and sensitivity, which renders sexual activity less pleasurable. By holding the user's remaining penile shaft skin in position to contact and cover the glans and inner skin, the present invention allows these parts to regain health.

Dissatisfied circumcised men have undertaken various methods to apply tension to the penile shaft skin in an effort to regrow enough skin to keep the glans covered. Thousands of men can testify that with a few years of gentle but persistent tension, new skin can be grown. Some of the methods used involve messy adhesive tapes which must be removed and re-applied daily, causing great discomfort to the practitioners of these methods. Sometimes the methods involve the wearing of uncomfortable elastic straps wrapped around parts of the body in order to apply tension to a device attached to the penile skin. Sometimes bulky weights are attached to a stretching device.

The present invention overcomes the drawbacks of tapes, straps, and weights to provide a means for a circumcised man to comfortably and discretely keep his glans covered in a protective sheath of his own skin. The invention also includes ancillary devices which provide the added benefit of tensioning the skin so a skin growth regimen can be followed even if straps, tapes, and weights cannot be tolerated by the circumcision victim.

### BRIEF SUMMARY OF THE INVENTION

This invention is a cone-shaped rubber shell (or conical retainer) which a circumcised man can use to hold his remaining penile shaft skin in a manner such that it covers his glans, just as if he still had a foreskin. Through careful selection of materials and novel geometry, this device succeeds at gripping the skin and retaining it over the glans without any form of adhesive. Thus, the device can be removed instantly for urination as needed. To apply the device, the user rolls the shaft skin of his flaccid penis away from his body until it envelopes his glans. Then he applies the invention to the rolled skin, like a little hat for the penis. The skin's tendency to elastically roll back to its original position combine with the geometry of the glans and the novel geometry and texture of the invention to bring about a state of equilibrium which holds the device in place.

Applying the device takes less than 5 seconds and removing the device takes less than one second. The device can be worn discretely under briefs in a work setting, and can be worn while the user sleeps (whether it stays put all night or not depends on the user's sleep patterns and the state of his penile skin). A primary advantage of this method is that the user's own skin (and not some device) is in contact with the glans and inner skin. No man-made device could impart the health benefits to these parts the way the user's own skin can.

The user can also add optional spacers—including a conical spacer and cylindrical spacers—between his penis and the conical retainer to afford a bit of skin tension. The skin responds to this tension by growing, and this system of devices can provide tension without the weights or straps as were required by prior methods. Eventually, the user may have enough skin to keep his glans covered without any device. But even if he does not choose to endure tension on his skin, the basic conical retainer device is so simple, comfortable, discrete, and—presumably—affordable that he can conceivably be content to use the device for the rest of his life. Also claimed are fixtures for manufacture of the devices.

### BRIEF DESCRIPTION OF THE SEVERAL VIEWS OF THE DRAWING

FIG. 1—depicts the basic conical retainer in pictoral, top, front, and section views.

FIG. 2—depicts a section view of a human penis with the conical retainer applied.

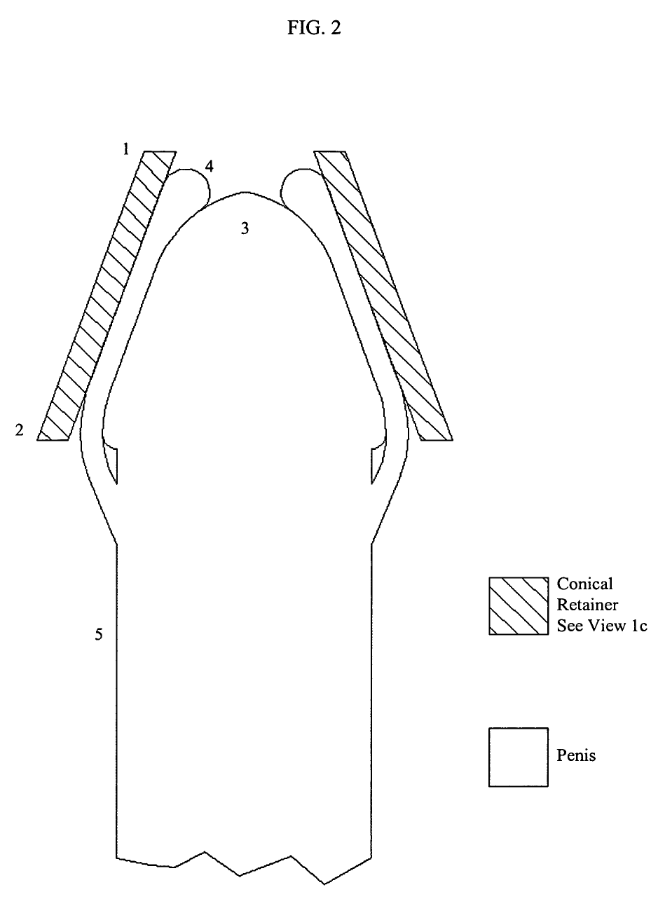

FIG. 3—depicts the female part of the mold for casting the conical retainer in pictoral, top, and front views.

FIG. 4—depicts the male part of the mold for casting the conical retainer in pictoral, top, and front views.

FIG. 5—depicts the male and female parts of the mold for casting the conical retainer, joined together, in pictoral, top, and front views.

FIG. 6—depicts the conical spacer in pictoral, top, front, and section views.

FIG. 7—depicts a cylindrical spacer in pictoral, top, front, and section views.

FIG. 8—depicts a section view of a human penis with the conical retainer, conical spacer, and cylindrical spacer applied.

FIG. 9—depicts the male and female parts of the mold for casting the conical spacer, joined together, in top and sectional views.

FIG. 10—depicts the three parts of the mold system for casting the cylindrical spacer—the cylinder cavity, the adjustable plug, and the male mold cap—joined together, in top, front, and sectional views.

### DETAILED DESCRIPTION OF THE INVENTION

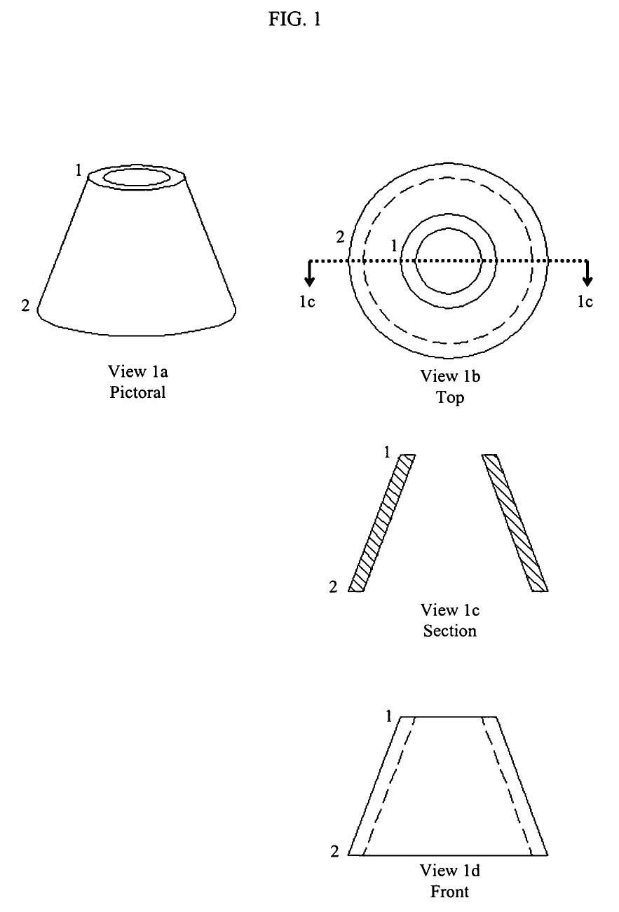

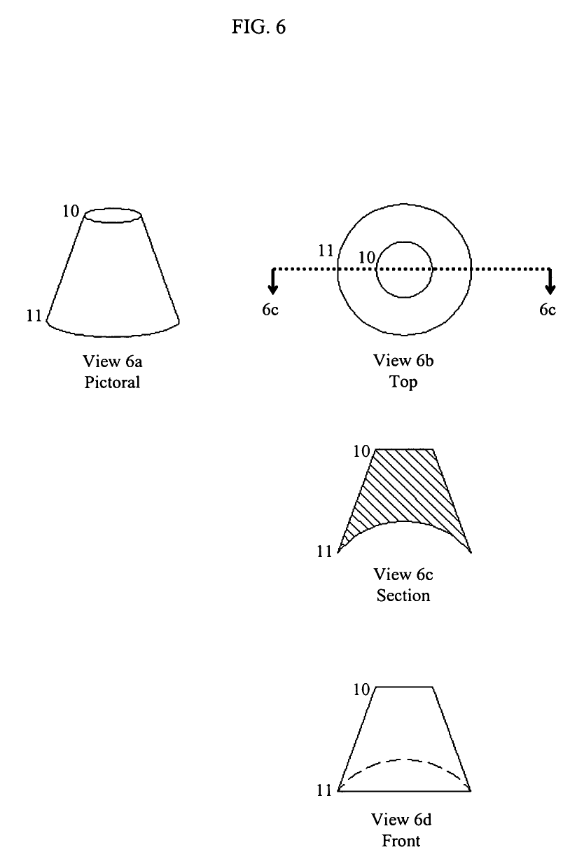

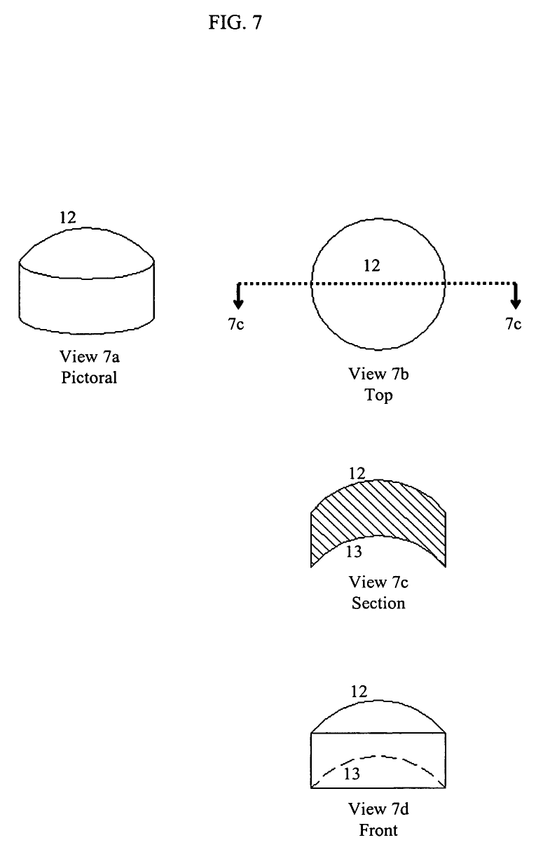

This invention includes three devices worn by an end user: a conical retainer (see FIG. 1), a conical spacer (see FIG. 6), a cylindrical spacer (see FIG. 7). All of these are cast of silicone rubber, for example Dow-Corning Silastic E, which is a food-grade, platinum-catalyzed, room-temperature-vulcanizing, heat-accelerated-cure compound. These properties make it ideal for a skin-contact product. Devices cast from Silastic E can be boiled to sterilize them as often as necessary. The cured durometer of 35-40 provides ideal tactile properties and strength, and the products can be manufactured affordably.

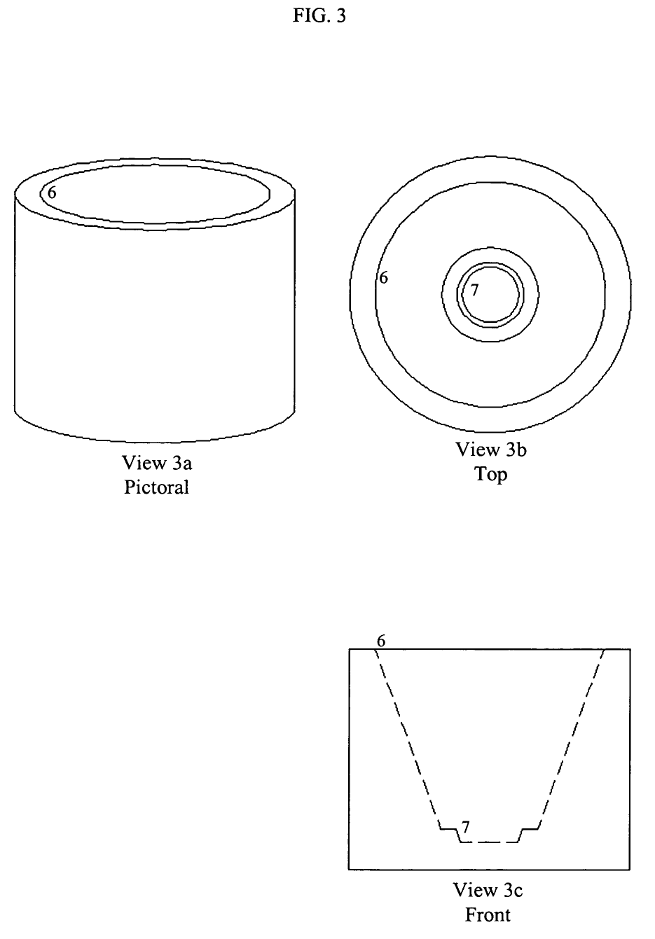

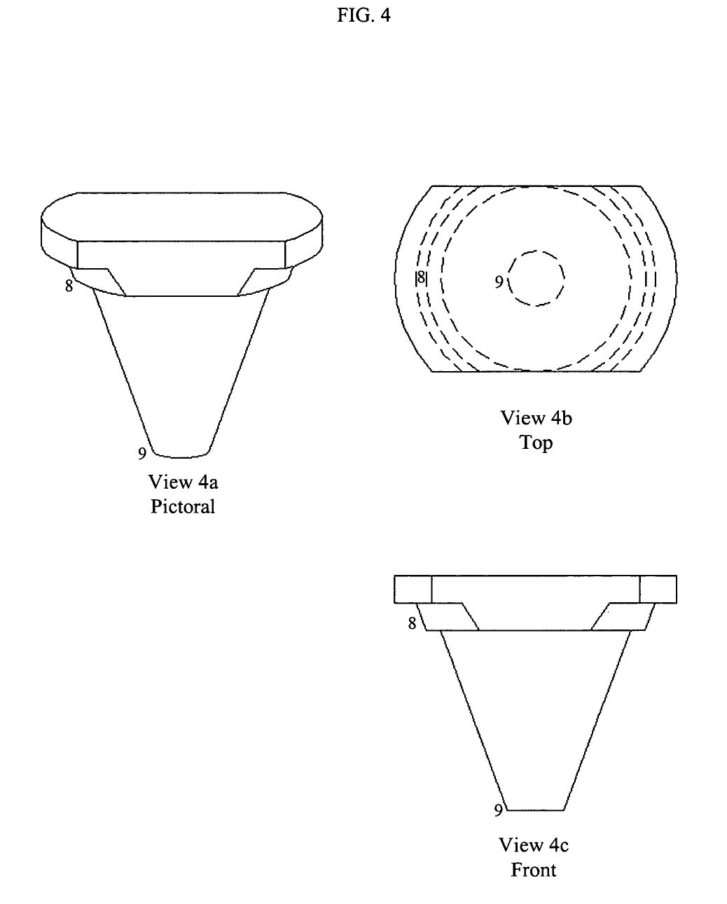

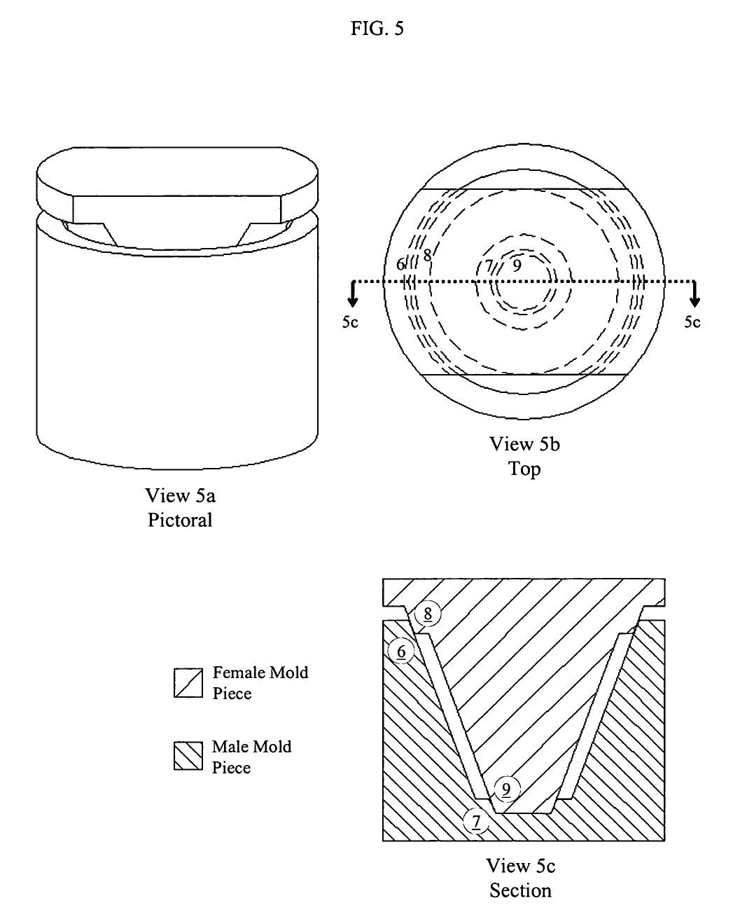

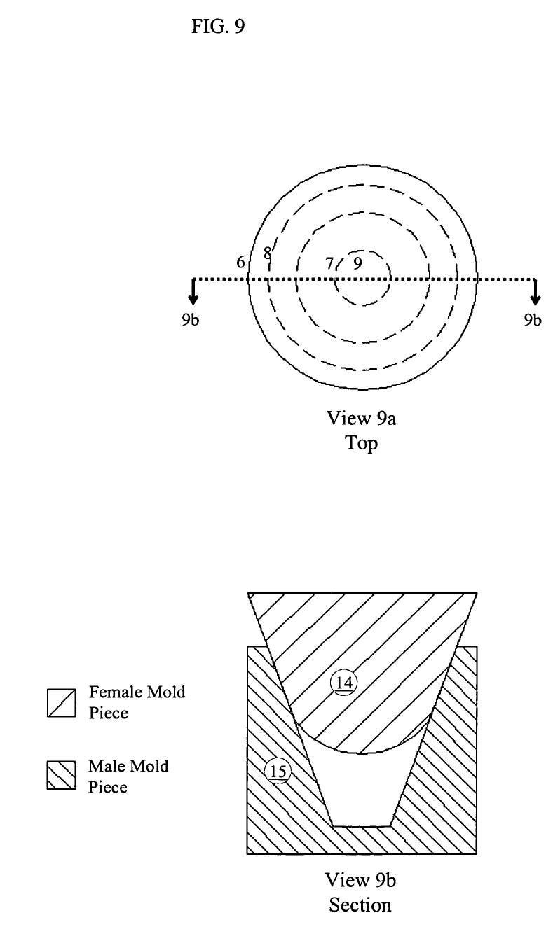

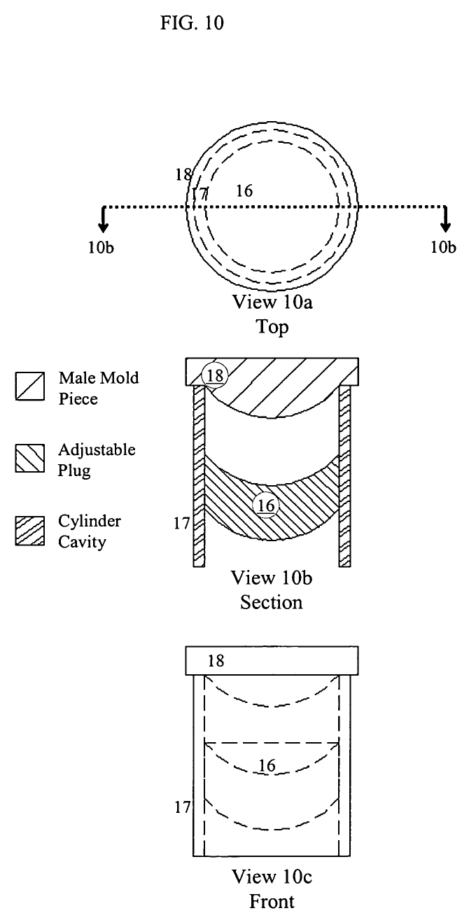

The invention also includes molding fixtures for making the conical retainer (see FIGS. 3-5), the conical spacer (see FIG. 9), and the cylindrical spacer (see FIG. 10). These molds can be created out of ABS plastic through the process of stereolithography. In each case, a measured mass of silicone is poured into the female half of the mold and then the male piece is applied over the top before the mold is placed in an oven for curing. Since the silicone does not bond the to the ABS, de-molding the parts is not a challenge. Note that in the case of the cylindrical spacer, the lower half of the mold consists of an outer cylindrical cavity 17 and an inner adjustable plug 16 which can slide to different heights to mold pieces of variable size. The plug can be made to stay in place by putting blocks beneath it.

The conical retainer (View 1a) is a rubber shell in the shape of a truncated cone. The diameter at the narrow end 1 is about 0.6 inches and at the wide end 2 it is about 1.5 inches. The cone walls are about 0.125 inches thick and slope at an angle of 20 degrees off vertical. The net altitude is about 1.25 inches. Of course the device can be made larger—with all the same angles—by merely pouring more silicone into the mold, to accommodate a larger-than-average penis.

The device stays in place on a user due to two forces. One is the friction between the interior of the device and the man's skin 4. The other force is the elastic tendency of the skin to roll back away from the glans 3, toward the shaft of the penis 5. Because rolling off the naturally tapered glans would require the skin to roll outward to clear the widest part of the glans (adjacent to the corona), the skin can not roll off the glans without getting wider. The invention prevents this because of friction. The elasticity of the skin tries to pull the skin wider, and friction with the device prevents the widening and draws the device more snugly toward the general direction of the base of the penis, and an equilibrium state is reached.

The open narrow end allows the user to see that the attempted application has produced an even distribution of accumulated skin. If the skin is observed to be wrinkled or unevenly distributed—which could cause discomfort—the device can be removed and re-applied in seconds.

The conical spacer (View 6a) is used in conjunction with the conical retainer. A user places the concave part at the wide end 11 of the spacer against his glans, then rolls his shaft skin over the glans and up to the narrow end 10 of the conical spacer before applying the conical retainer.

The conical spacer is a truncated cone with a diameter at the narrow end of 0.5 inches, and a diameter at the wide end of 1.2 inches. It has the same 20 degree (off vertical) wall angle as the conical retainer. It also has a concave base to comfortably interface with the glans. The spheroidal concavity has a radius of 0.75 inches.

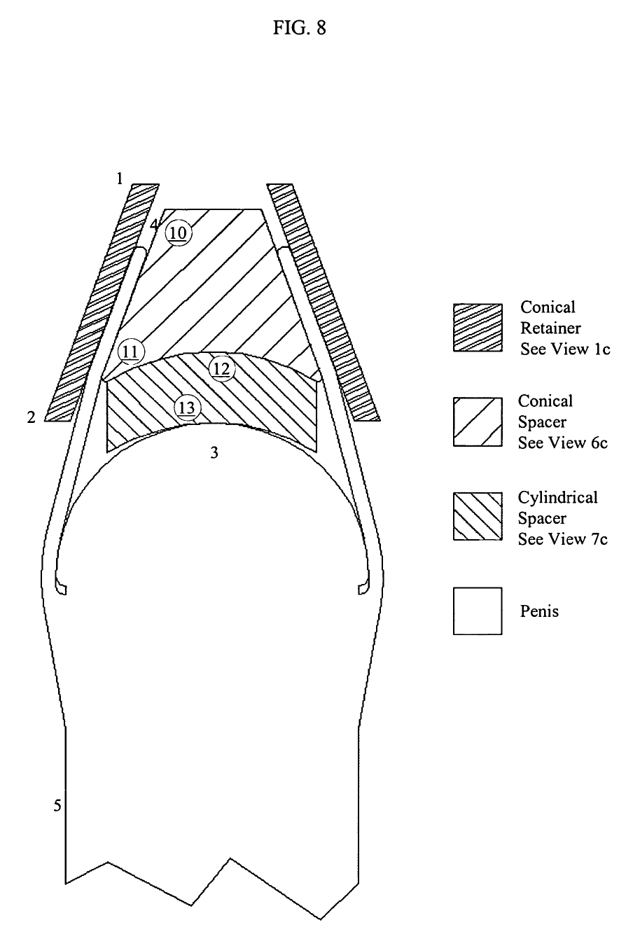

The cylindrical spacer has a diameter of 1.2 inches. The top 12 is spheroidally convex to interface with the base of the conical spacer. The base 13 is spheroidally concave to interface with the glans, as shown in FIG. 8. A user places the concave side of the cylindrical spacer against his glans, places the conical spacer against the cylindrical spacer, and then rolls his shaft skin over the glans and spacers up to the narrow end of the conical spacer before applying the conical retainer. The cylindrical spacer would typically be about 0.5 inches tall, but the same principal applies for cylinder heights of 0 through 1 inch, or more. All these sizes can be molded in the apparatus depicted in FIG. 10.

The molds for the conical retainer (FIGS. 3-5) are made of ABS plastic. The female part (View 3a) has a generally concave form as shown with a special aligning recess 7 in the bottom. The silicone is poured in and then the male part is pushed down through the opaque compound until the point of the female tool aligns with and enters the recess. At that point, the female tool also supports the male tool at the wide end 6 of the female tool.

The male tool for molding the conical retainer is generally conical and very smooth to form the inside of the conical retainer. It has a blunt end 9 to mate with the recess in the female tool and a flare 8 near the top to rest on the top edge of the female tool. When used together (View 5a), the male and female tool have an internal clearance where the conical retainer can be cast in varying sizes, and the two mold parts have a gap (near the shoulder 8 of the female tool) between them which allows them to be pried apart once the silicone has cured. The top of the female mold also features flattened sides which provide for a view into the mold for checking material distribution. The flats also provide a means to exert torsion to aid in de-molding the cured parts.

The molds for the conical spacer (FIG. 9) are also made of ABS plastic. The female part 15 has a generally concave form as shown. The measured silicone is poured in and then the male part 14 is pushed down into the compound until the sloped surfaces of the male tool rest against the surfaces of the female tool.

The male tool for the conical spacer mold is generally conical with a pointy end formed into a spheroidal shape with radius 0.75 inches to form the concave recess in the conical spacer.

The mold for the cylindrical spacer is a cylindrical cavity with an inside diameter of 1.2 inches and an adjustable plug fitting snugly within it. The plug has a convex spheroidal radius of 0.75 inches to mold the concave end of the cylindrical spacer. The plug can be moved up and down within the cylindrical cavity to allow for molding parts of any thickness. After the measured silicone is poured in, the male mold piece 18 is applied as shown to cast the concave part of the cylindrical spacer.

1. A device for holding the foreskin of a circumcised penis in a fully extended glans-covering state, consisting of:
a. A conical retainer which is a tapered concave shell in the shape of a truncated cone (FIG. 1a), having:
i. a smooth interior, and
ii. uniform wall thickness of 0.125 inch, and
iii. an opening at the narrow end of an inside diameter 0.6 inches, and
iv. an opening at the wide end of an inside diameter 1.5 inches, and
v. a net altitude of 1.25 inches, giving rise to wall angles 20 degrees off vertical, and made of
vi. Dow Corning Silastic E Silicone Rubber or similar skin-safe compound yielding a cured durometer of roughly 40, so that the device will stretch no more than 10% under typical tensions generated by adult hands.
b. The device as described is inconspicuous even when worn under shorts or swimming trunks.

2. The device of claim 1 with the same angles, wall thickness, and small opening size, but with a larger wide opening and altitude to accommodate a larger than average penis.

3. A 2-piece ABS mold in which the devices of claim 1 and 2 can be cast, consisting of:
a. A female mold which has a concave conical opening with a shallow recess centered at the bottom.
b. A male mold which has a tapered convex protuberance in the shape of a truncated cone, having:
i. a flattened tip of diameter 0.5 inches, and
ii. wall angles 20 degrees off vertical, up to a stepped out section at an altitude of 1.5 inches, where
iii. there are two flared sections corresponding to the 0.125 inch wall thickness of the device of claim 1, which allow for centering of this male mold within the concavity of the female mold of part a, while affording a view down into the void between the two pieces.
c. These two parts are used by pouring perhaps 11 g of silicone casting compound into the conical opening, then pressing the protuberance into the conical opening until the tip is in the shallow recess and the flared sections are against the walls of the conical opening. The compound is allowed to harden and then the two parts are pried apart so the device of claim 1 can be removed. To make the device of claim 2, use perhaps an additional 5 g of casting compound at the pouring step.

4. A conical spacer, which is a complementary device to the device of claim 1, with a solid conical body, to be used between the device of claim 1 and the glans of the user, to act as a spacer which will increase the tension on the penile shaft skin drawn up over the glans and spacer, without an external tugging apparatus. The base of the device is concave with a spheroidal contour to comfortably interface with the glans of the user.

5. A cylindrical spacer, which is a complementary device to the device of claim 4, consisting of a solid cylindrical body of altitude perhaps 0.5 inches, to be used between the device of claim 4 and the glans of the user, to act as an extra spacer which will further increase the tension on the penile shaft skin drawn up over the glans and spacers, without an external tugging apparatus. The device has a spheroidal concavity at one end (to interface with the glans of the user), and a complementary spheroidal convexity at the other end (to interface with the concavity of the device of claim 4).

6. A 2-piece set of molds in which the conical spacer device of claim 4 can be cast, consisting of:
a. A female mold with a concave conical opening with a flat bottom, and
b. A separate male mold with a conical and semi-spheroidal convex protuberance.

7. A 3-piece set of molds in which the device of claim 5 can be cast, consisting of:
a. A hollow cylindrical cavity with open ends, and
b. A concave semi-spheroidal adjustable plug insert with cylindrical outer walls, which can be positioned within the device of part a, at various distances from the open end, and
c. A male mold piece forming a convex semi-spheroidal cap.

8. A method of keeping the glans covered by a circumcised wearer's own shaft skin, involving the rolling of shaft skin away from the body until it covers the glans, and the application of a conical shell to comfortably and discretely retain the skin in this glans-covering state without adhesive.
